(19) United States
(12) Patent Application Publication
(10) Pub. No.: US 2024/0307682 A1
(43) Pub. Date: Sep. 19, 2024

(54) IMPLANTABLE MEDICAL DEVICE LEAD WITH SHOCKING ELECTRODE

(71) Applicant: Pacesetter, Inc., Sylmar, CA (US)

(72) Inventors: Gene A. Bornzin, Los Angeles, CA (US); Diane M. Muff, King, WA (US); Benjamin F. James, IV, Sylmar, CA (US); Zoltan Somogyi, Ventura, CA (US)

(21) Appl. No.: 18/600,991

(22) Filed: Mar. 11, 2024

Related U.S. Application Data
(60) Provisional application No. 63/490,050, filed on Mar. 14, 2023.

(51) Int. Cl. A61N 1/05; A61N 1/39

(52) U.S. Cl. CPC A61N 1/0563; A61N 1/3956

(57) ABSTRACT

A lead of an implantable medical device (IMD) includes a shocking electrode configured to deliver high-voltage shocks for defibrillation therapy. The shocking electrode includes a base structure that has an oblong cross-sectional shape with a first side and a second side that is opposite the first side. The base structure has a set of grooves defined along the first side. The grooves in the set are configured to receive a cable assembly that is placed into the grooves in a side-loading direction.

### CROSS-REFERENCE TO RELATED APPLICATIONS

This application is a non-provisional conversion of, and claims priority to, U.S. Provisional Patent Application No. 63/490,050, which was filed Mar. 14, 2023, and the entire disclosure of which is incorporated herein by reference.

### FIELD

Embodiments of the present disclosure generally relate to leads of implantable medical devices (IMDs) with one or more electrodes designed to deliver electrical stimulation for defibrillation therapy. In one or more embodiments, the leads may be designed for subcutaneous placement within a patient.

### BACKGROUND

Some IMDs include circuitry that monitors a patient's heart rhythm to detect arrythmias, such as ventricular tachycardia and/or atrial fibrillation. In response to detecting an arrythmia, the same or a different IMD may deliver a powerful electrical shock to defibrillate the heart. For example, implantable cardioverter defibrillators (ICDs) are IMDs which include a battery-operated pulse generator that generates high voltage shocks and at least one lead extending from the generator to deliver the shocks. Some ICD leads are intra-cardiac and/or transvenous, such that the leads are introduced on or in heart tissue or in surrounding blood vessels. Some ICD systems are subcutaneous and deliver defibrillation therapy without any intra-cardiac or transvenous leads. The subcutaneous ICDs (S-ICD) include at least one subcutaneous lead extending from the generator. The subcutaneous lead is implanted below the skin but outside of the cardiac tissue and blood vessels. The subcutaneous lead may be implanted along an exterior of the rib cage. The lead may be proximate to the sternum. S-ICD systems eliminate risks associated with transvenous and/or intra-cardiac implanted leads, such as infections and lead failures that may require surgical intervention.

A drawback of known leads that deliver shocks for defibrillation therapy is manufacturing complexity. The lead may include electrical cables that connect one or more electrodes to the pulse generator. Typically, the electrical cables are axially loaded into an interior of a shocking electrode through one or more openings at a proximal end of the shocking electrode. It may be difficult to align the cables with the opening(s) and/or to advance the cables a sufficient depth into the shocking electrode. For example, some cables may need to be fed through a full length of the shocking electrode. It may also be difficult to access the cables, once in the desired position, for affixing the cables to the shocking electrode and/or other electrodes adjacent to the shocking electrode. For example, some shocking electrodes may be formed with apertures through side walls to permit spot-welding a cable within the interior of the shocking electrode. Forming the shocking electrode with such apertures and routing a cable through the end of the shocking electrode to reach the apertures may be relatively complex.

A need remains for IMD leads with shocking electrodes that are less complex and easier to manufacture than known IMD leads with shocking electrodes.

### SUMMARY

In accordance with an embodiment, a lead of an implantable medical device (IMD) is provided. The lead includes a shocking electrode configured to deliver high-voltage shocks for defibrillation therapy. The shocking electrode includes a base structure that has an oblong cross-sectional shape with a first side and a second side that is opposite the first side. The base structure has a set of grooves defined along the first side. The grooves in the set are configured to receive a cable assembly that is placed into the grooves in a side-loading direction.

Optionally, the lead also includes the cable assembly. The cable assembly may include multiple cables and a tubular member that collectively surrounds the cables. The set of grooves may include a recess to accommodate the tubular member and multiple slots extending from the recess to accommodate the cables. The tubular member may include a neck and a flange that has a greater diameter than the neck. The recess in the set of grooves may include a narrow segment and a broad segment, with the narrow segment disposed between a proximal end of the base structure and the broad segment. The narrow segment may be configured to receive the neck of the tubular member, and the broad segment may be configured to receive the flange of the tubular member. The flange may have a greater diameter than the narrow segment to axially secure the cable assembly to the shocking electrode.

Optionally, the base structure is defined by multiple brick segments that are discrete and mechanically connected to one another in a line. The set of grooves is defined along the first side of at least two of the brick segments. The brick segments may be electrically conductive and electrically connected to one another. A power cable of the cables of the cable assembly may be welded to one of the brick segments to establish a conductive pathway from a pulse generator of the IMD to the shocking electrode. The set of grooves may be a first set of grooves, and the base structure may include a second set of one or more grooves defined along the first side and spaced apart from the first set of grooves. The shocking electrode may include one or more support wires disposed within the second set of grooves and affixed to the brick segments to secure the brick segments to one another in the line.

Optionally, the base structure is an insulative body that extends from a proximal end of the shocking electrode to a distal end of the shocking electrode. The shocking electrode may include an electrically conductive layer disposed along the second side of the insulative body. The electrically conductive layer may be electrically connected to a power cable of the cable assembly to establish a conductive pathway from a pulse generator of the IMD to the electrically conductive layer to deliver the high-voltage shocks for the defibrillation therapy. The electrically conductive layer may include one or more metal plates affixed to the second side of the insulative body.

Optionally, the shocking electrode is a distal shocking electrode, and the lead further comprises a proximal shocking electrode. Optionally, the shocking electrode includes one or more cover plates that cover a portion of the cable assembly that is within the set of grooves, such that the portion of the cable assembly is disposed between the one or more cover plates and the second side of the base structure. Optionally, the shocking electrode includes an overmold material on the first side of the base structure and a portion of the cable assembly that is within the set of grooves. The overmold material may conform to a contour of the portion of the cable assembly and encase the portion of the cable assembly between the base structure and the overmold material.

In accordance with an embodiment, a method of producing a lead for an implantable medical device (IMD) is provided. The method includes forming a set of grooves along a first side of a base structure of a shocking electrode configured to deliver high-voltage shocks for defibrillation therapy. The base structure has an oblong cross-section shape with a second side that is opposite the first side. The method includes depositing a portion of a cable assembly into the grooves of the set in a side-loading direction.

Optionally, the cable assembly includes multiple cables and a tubular member that collectively surrounds the cables. The set of grooves includes a recess and multiple slots extending from the recess. Depositing the portion of the cable assembly into the grooves includes placing the tubular member into the recess and placing the cables into different corresponding slots of the set of grooves.

Optionally, the method includes assembling the base structure by mechanically connecting a plurality of discrete brick segments together in a line. The set of grooves is formed along the first side of at least two of the brick segments. In an example, the brick segments are electrically conductive and electrically connected to one another, and the method includes welding a power cable of the cable assembly to one of the brick segments to establish a conductive pathway from a pulse generator of the IMD to the shocking electrode. In an example, the set of grooves is a first set of grooves, and the base structure further includes a second set of one or more grooves defined along the first side and spaced apart from the first set of grooves. The method includes depositing one or more support wires in the side-loading direction into the second set of grooves and affixing the one or more support wires to the brick segments to secure the brick segments to one another in the line.

Optionally, the base structure is an insulative body that extends from a proximal end of the shocking electrode to a distal end of the shocking electrode. The method may include applying an electrically conductive layer along the second side of the insulative body, and affixing a power cable of the cable assembly to the electrically conductive layer to establish a conductive pathway from a pulse generator of the IMD to the electrically conductive layer to deliver the high-voltage shocks for the defibrillation therapy. Applying the electrically conductive layer may include affixing one or more metal plates to the second side of the insulative body.

Optionally, the method includes covering the portion of the cable assembly that is within the set of grooves with one or more cover plates such that the portion of the cable assembly is disposed between the one or more cover plates and the second side of the base structure. Optionally, the method includes applying an overmold material on the first side of the base structure and the portion of the cable assembly that is within the set of grooves. The overmold material conforms to a contour of the cable assembly and encases the cable assembly between the base structure and the overmold material. Optionally, the method includes implanting the lead such that the shocking electrode is disposed in a subcutaneous location within the patient.

In accordance with an embodiment, an implantable medical device (IMD) is provided that includes a pulse generator and a lead. The lead includes a lead body, a cable assembly, and a shocking electrode. The cable assembly electrically and mechanically connects the lead body to the shocking electrode, and the lead body extends to the pulse generator. The pulse generator powers the shocking electrode, via the lead body and the cable assembly, to deliver high-voltage shocks for defibrillation therapy. The shocking electrode includes a base structure that has an oblong cross-sectional shape including a first side and a second side that is opposite the first side. The base structure includes a set of grooves defined along the first side. A portion of the cable assembly is loaded into the grooves of the set in a side-loading direction.

### BRIEF DESCRIPTION OF THE DRAWINGS

FIG. 1 illustrates a graphical representation of an implantable medical device (IMD) that is configured to apply defibrillation therapy in accordance with embodiments herein.

FIG. 2 shows a block diagram of an IMD that is configured to be implanted into a patient.

FIG. 3 is an exploded view of a portion of a lead of an IMD according to an embodiment.

FIG. 4 is a side view of an electrode module of the lead of FIG. 3 according to an embodiment.

FIG. 5 is a cross-sectional view of the electrode module according to an embodiment.

FIG. 6 is a plan view showing the lead of FIG. 3 in a partially assembled state.

FIG. 7 is an enlarged view of a portion of the lead showing a recess in the electrode module and a tubular member of a cable assembly according to an embodiment.

FIG. 8 is a plan view showing the lead of FIG. 3 in a fully assembled state according to an embodiment.

FIG. 9 is a cross-sectional view of the lead including an overmold material on the electrode module according to an embodiment.

FIG. 10 is a plan view of the electrode module according to an alternative embodiment.

FIG. 11 is a cross-sectional view of the lead including the electrode module of FIG. 10.

FIG. 12 is a flow chart of a method for producing a lead for an IMD according to an embodiment.

### DETAILED DESCRIPTION

It will be readily understood that the components of the embodiments as generally described and illustrated in the figures herein, may be arranged and designed in a wide variety of different configurations in addition to the described example embodiments. Thus, the following more detailed description of the example embodiments, as represented in the figures, is not intended to limit the scope of the embodiments, as claimed, but is merely representative of example embodiments.

Reference throughout this specification to “one embodiment” or “an embodiment” (or the like) means that a particular feature, structure, or characteristic described in connection with the embodiment is included in at least one embodiment. Thus, appearances of the phrases “in one embodiment” or “in an embodiment” or the like in various places throughout this specification are not necessarily all referring to the same embodiment.

Furthermore, the described features, structures, or characteristics may be combined in any suitable manner in one or more embodiments. In the following description, numerous specific details are provided to give a thorough understanding of embodiments. One skilled in the relevant art will recognize, however, that the various embodiments can be practiced without one or more of the specific details, or with other methods, components, materials, etc. In other instances, well-known structures, materials, or operations are not shown or described in detail to avoid obfuscation. The following description is intended only by way of example, and simply illustrates certain example embodiments.

The methods described herein may employ structures or aspects of various embodiments (e.g., systems and/or methods) discussed herein. In various embodiments, certain operations may be omitted or added, certain operations may be combined, certain operations may be performed simultaneously, certain operations may be performed concurrently, certain operations may be split into multiple operations, certain operations may be performed in a different order, or certain operations or series of operations may be re-performed in an iterative fashion. It should be noted that, other methods may be used, in accordance with an embodiment herein. Further, wherein indicated, the methods may be fully or partially implemented by one or more processors of one or more devices or systems. While the operations of some methods may be described as performed by the processor(s) of one device, additionally, some or all of such operations may be performed by the processor(s) of another device described herein.

Embodiments may be implemented in connection with one or more implantable medical devices (IMDs). Non-limiting examples of IMDs include one or more of neurostimulator devices, implantable leadless monitoring and/or therapy devices, and/or alternative implantable medical devices. For example, the IMD may represent a cardiac monitoring device, pacemaker, cardioverter, cardiac rhythm management device, defibrillator, neurostimulator, leadless monitoring device, leadless pacemaker and the like. For example, the IMD may include one or more structural and/or functional aspects of the device(s) described in U.S. Pat. No. 9,333,351 “Neurostimulation Method And System To Treat Apnea” and U.S. Pat. No. 9,044,610 “System And Methods For Providing A Distributed Virtual Stimulation Cathode For Use With An Implantable Neurostimulation System”, which are hereby incorporated by reference.

Additionally or alternatively, the IMD may be a subcutaneous IMD (e.g., a S-ICD) that includes one or more structural and/or functional aspects of the device(s) described in U.S. application Ser. No. 17/804,041, titled “Method And Implantable Medical Device For Reducing Defibrillation Impedance” and filed May 25, 2022; U.S. application Ser. No. 15/973,195, titled “Subcutaneous Implantation Medical Device With Multiple Parasternal-Anterior Electrodes” and filed May 7, 2018; U.S. application Ser. No. 15/973,219, titled “Implantable Medical Systems And Methods Including Pulse Generators And Leads” and filed May 7, 2018; U.S. application Ser. No. 15/973,249, titled “Single Site Implantation Methods For Medical Devices Having Multiple Leads”, filed May 7, 2018, which are hereby incorporated by reference in their entireties. Further, one or more combinations of IMDs may be utilized from the above incorporated patents and applications in accordance with embodiments herein.

All references, including publications, patent applications and patents, cited herein are hereby incorporated by reference to the same extent as if each reference were individually and specifically indicated to be incorporated by reference and were set forth in its entirety herein.

### Terms

The terms “cardiac activity signal”, “cardiac activity signals”, “CA signal” and “CA signals” (collectively “CA signals”) are used interchangeably throughout to refer to measured signals indicative of cardiac activity by a region or chamber of interest. For example, the CA signals may be indicative of impedance, electrical or mechanical activity by one or more chambers (e.g., left or right ventricle, left or right atrium) of the heart and/or by a local region within the heart (e.g., impedance, electrical or mechanical activity at the AV node, along the septal wall, within the left or right bundle branch, within the purkinje fibers). The cardiac activity may be normal/healthy or abnormal/arrhythmic. An example of CA signals includes EGM signals. Electrical-based CA signals refer to an analog or digital electrical signal recorded by two or more electrodes, where the electrical signals are indicative of cardiac activity. Heart sound (HS) based CA signals refer to signals output by a heart sound sensor such as an accelerometer, where the HS based CA signals are indicative of one or more of the S1, S2, S3 and/or S4 heart sounds. Impedance based CA signals refer to impedance measurements recorded along an impedance vector between two or more electrodes, where the impedance measurements are indicative of cardiac activity.

The terms “high-voltage shock” and “HV shock” refer to defibrillation stimulus delivered at an energy level sufficient to terminate a defibrillation episode in a heart, wherein the energy level is defined in Joules to be 40J or more and/or the energy level is defined in terms of voltage to be 750V or more.

The term “defibrillation threshold” and acronym “DFT” refer to a minimum amount of energy needed to be delivered in a high-voltage shock of defibrillation therapy in order to return a heart to a normal rhythm from a condition in which the heart is experiencing a fibrillation dysrhythmia episode.

The term “oblong” as used herein refers to elongated shapes that are longer in at least one dimension than another dimension, such that the oblong shapes are not circular/cylindrical or square/cubic. The longest dimension of a cross-sectional shape is referred to herein as a “major dimension,” and a shorter dimension of the cross-sectional shape is referred to as a “minor dimension.” The minor dimension may be perpendicular to the major dimension.

The terms “processor,” “a processor”, “one or more processors” and “the processor” shall mean one or more processors. The one or more processors may be implemented by one, or by a combination of more than one implantable medical device, a wearable device, a local device, a remote device, a server computing device, a network of server computing devices and the like. The one or more processors may be implemented at a common location or at distributed locations. The one or more processors may implement the various operations described herein in a serial or parallel manner, in a shared-resource configuration and the like.

The term “subcutaneous” shall mean below the skin, but not intravenous. For example, a subcutaneous lead and/or electrode is not located in a chamber of the heart, in a vein on the heart, or in the lateral or posterior branches of the coronary sinus. A subcutaneous lead and/or electrode may be located between the skin and the rib cage, or within an intercostal area between two ribs of the rib cage. The rib cage collectively refers to the ribs, sternum, and thoracic vertebrae. Subcutaneous placement is external of (and does not include) the substernal space, where the substernal space is defined between the undersurface of the rib cage and the pericardium or outer portion of the heart.

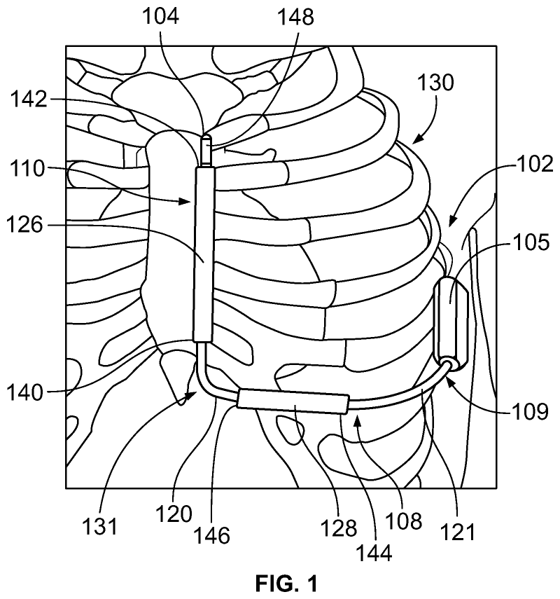

FIG. 1 illustrates a graphical representation of an implantable medical device (IMD) 102 that is configured to apply defibrillation therapy in accordance with embodiments herein. The IMD 102 in the illustrated embodiment is a subcutaneous implantable medical device (SIMD) that is configured to be implanted in a subcutaneous area exterior to the heart. The SIMD 102 includes a pulse generator 105 and at least one lead 120 that is operably coupled to the pulse generator 105. The “at least one lead” is hereinafter referred to as “the lead.” Nevertheless, it should be understood that the term, “the lead,” may mean only a single lead or may mean more than one single lead. The lead 120 includes a lead body 121 that is mechanically connected to the pulse generator 105 and extends from the pulse generator 105 to a distal tip 104 of the lead 120.

The pulse generator 105 includes a housing that contains power circuitry and energy storage devices for generating high-voltage shocks (HV shocks) for defibrillation therapy. The housing may be electrically conductive to form or constitute an electrode, referred to as the “CAN” electrode. The pulse generator 105 may be subcutaneously implanted within a pocket at a mix-axillary position along a portion of the rib cage 130 of the patient.

The lead 120 may be subcutaneously implanted. In particular embodiments, the SIMD 102 is an entirely or fully subcutaneous SIMD. The SIMD may not include a transvenous lead. The lead 120 in the illustrated embodiment includes a first or proximal segment 108 that extends from the pulse generator 105 along an inter-costal area between ribs. The lead 120 has a proximal end 109 that mechanically couples to the pulse generator 105, and electrically connects to the pulse generator 105 to establish conductive path(s) to the electrodes of the lead 120. The proximal segment 108 may be laterally oriented to extend along an anterior axillary area of the rib cage 130. The lead 120 has a second or distal segment 110 that extends from the proximal segment 108 to the distal tip 104. The distal segment 110 may extend along the sternum (e.g., over the sternum or parasternally within one to three centimeters from the sternum). The intersection between the distal and proximal segments 108, 110 may be located proximate to the xiphoid process of the patient.

The lead 120 includes at least one electrode that is electrically connected to the pulse generator 105 and delivers the HV shocks for defibrillation therapy. In the illustrated embodiment, the electrodes include a first or primary electrode 126 disposed along the distal segment 110 and a second or secondary electrode 128 disposed along the proximal segment 108. The electrodes 126, 128 are referred to herein as shocking electrodes. In an embodiment, when the pulse generator 105 generates a HV shock, the pulse generator 105 supplies electrical power to both of the shocking electrodes 126, 128, and both shocking electrodes 126, 128 may deliver the HV shocks based on the received electrical power. The shocking electrodes 126, 128 may concurrently deliver the HV shocks to different target areas of the heart.

The shocking electrodes 126, 128 are spaced apart from each other along the length of the lead 120 by a gap segment 131 of the lead body 121. The gap segment 131 may be proximate to the xiphoid process. The primary electrode 126 may be positioned along an anterior region of the chest, and the secondary electrode 128 may laterally extend between the primary electrode 126 and the pulse generator 105. The shocking electrodes 126, 128 may be subcutaneously positioned at a level that aligns with the heart of the patient for providing a sufficient amount of energy for defibrillation.

The primary electrode 126 may be oriented transverse to an orientation of the secondary electrode 128 when in the implanted position as shown in FIG. 1. For example, the primary electrode 126 has a first orientation extending from a proximal end 140 of the electrode 126 to a distal end 142 of the electrode 126 (defined along the length of the lead 120 relative to the pulse generator 105). The first orientation may be generally parallel to the midsternal line of the patient. The secondary electrode 128 has a second orientation extending from a proximal end 144 of the electrode 128 to a distal end 146 of the electrode 128. Optionally, the orientation of the secondary electrode 128 may define an angle between about 60 degrees and 120 degrees (e.g., such as 70 degrees to 110 degrees) relative to the orientation of the primary electrode 126. Due to the orientation, the lead 120 may be referred to as an L-shaped lead. The primary electrode 126 may be referred to as a parasternal electrode. The secondary electrode 128 may be referred to as a transverse electrode.

In an alternative embodiment, the SIMD 102 may lack the secondary electrode 128. For example, the proximal segment 108 may not have any shocking electrodes. The primary electrode 126 may be the only shocking electrode on the lead 120 that delivers the HV shocks supplied from the pulse generator 105.

In an embodiment, the lead 120 may include one or more sensing electrodes 148 to detect far field electrogram signals. The sensing electrode(s) 148 may collect subcutaneous cardiac activity (CA) signals in connection with multiple cardiac beats. In the illustrated embodiment, one sensing electrode 148 is disposed at the distal tip 104 of the lead 120. The SIMD 102 may process the CA signals to detect arrhythmias, such as ventricular tachycardia and/or atrial fibrillation. If an arrhythmia is detected, the SIMD 102 may automatically take one or more actions depending on characteristics of the arrythmia, such as type and severity. The actions may include delivering one or more electrical HV shocks (e.g., shock pulses) via the shocking electrodes 126, 128 in an attempt to achieve cardioversion. Optionally, another IMD may be implanted within the heart, such as a leadless pacemaker. The SIMD 102 may be configured to communicate with the other intra-cardiac IMD. For example, the intra-cardiac IMD may signal to the SIMD 102 when an arrythmia is detected for the SIMD 102 to deliver the HV shocks in response to receiving the signal.

In an embodiment, at least one of the shocking electrodes 126, 128 of the lead 120 has an oblong cross-sectional area, which may reduce the shocking impedance, as described in U.S. application Ser. No. 17/804,041. A major dimension of the oblong shocking electrode 126, 128 represents the largest or broadest dimension of the cross-sectional area. The cross-sectional area of the oblong shocking electrode 126, 128 may include both the major dimension and a minor dimension that is perpendicular (i.e., orthogonal) to the major dimension. The minor dimension is smaller than the major dimension. The minor dimension may represent the smallest or narrowest dimension of the cross-sectional area, which is perpendicular to the major dimension. An aspect ratio of the major dimension to the minor dimension may be at least 2:1. The oblong cross-sectional shape may be a racetrack shape with two opposite planar sides and two opposite curved sides between the two opposite planar sides. The shocking electrode 126, 128 may have other oblong shapes, such as a rectangle with rounded corners, an oval (e.g., elliptical, egg-shaped, etc.), or the like. Optionally, the major dimension may be at least 10 French (F), which may achieve a low shocking impedance and maintain a low DFT, even in the presence of fat.

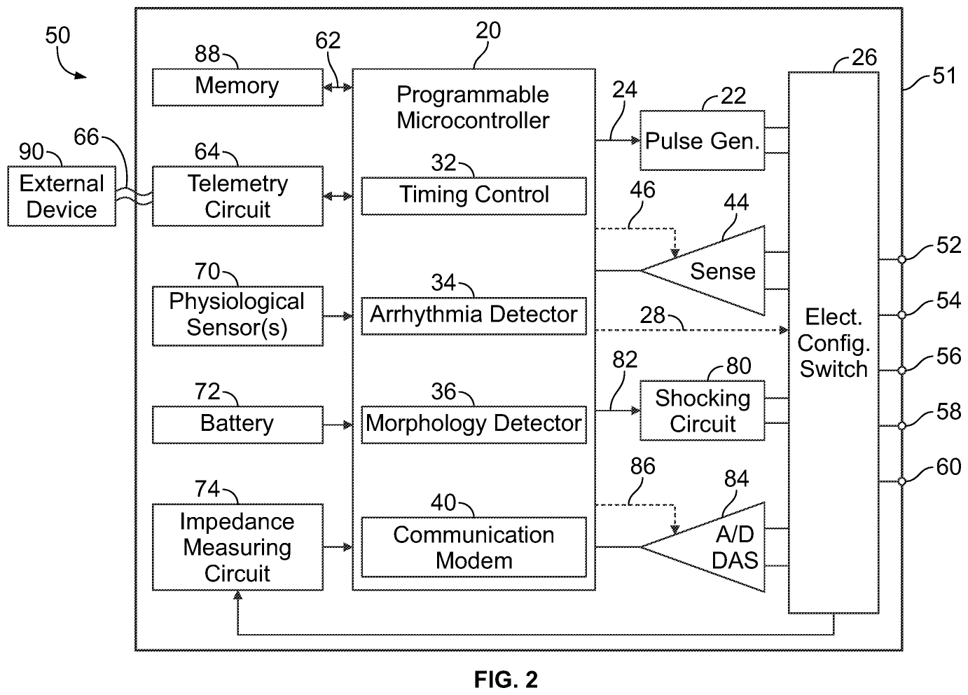

FIG. 2 shows a block diagram of an IMD 50 that is configured to be implanted into a patient. The IMD 50 may represent the pulse generator 105 of the SIMD 102 shown in FIG. 1. The IMD 50 may be implemented to monitor ventricular activity alone, or both ventricular and atrial activity through sensing circuit. The IMD 50 may treat both fast and slow arrhythmias with stimulation therapy, including cardioversion, pacing stimulation, an implantable cardioverter defibrillator, suspend tachycardia detection, tachyarrhythmia therapy, and/or the like.

The IMD 50 has a device case (or housing) 51 to hold the electronic/computing components. The case 51 (which can also be referred to as the “housing,” “can,” “encasing,” or “case electrode”) may be programmably selected to function as an electrode for certain sensing modes. Case 51 further includes a connector (not shown) with at least one terminal 52 and optionally additional terminals 54, 56, 58, 60. The terminals may be connected to electrodes that are located in various locations within and about the heart. The type and location of each electrode may vary. For example, the electrodes may include various combinations of ring, tip, coil, shocking electrodes, and the like.

The IMD 50 includes a programmable microcontroller 20 that controls various operations of the IMD 50, including cardiac monitoring and stimulation therapy. Microcontroller 20 includes a microprocessor (or equivalent control circuitry), RAM and/or ROM memory, logic and timing circuitry, state machine circuitry, and I/O circuitry. Microcontroller 20 includes an arrhythmia detector 34 that is configured to cardiac activity data to identify potential AF episodes as well as other arrhythmias (e.g., Tachycardias, Bradycardias, Asystole, etc.).

An electrode configuration switch 26 is optionally provided to allow selection of different electrode configurations under the control of the microcontroller 20. The electrode configuration switch 26 may include multiple switches for connecting the desired electrodes to the appropriate I/O circuits, thereby facilitating electrode programmability. The switch 26 is controlled by a control signal 28 from the microcontroller 20. Optionally, the switch 26 may be omitted and the I/O circuits directly connected to a housing electrode.

The IMD 50 further includes a chamber pulse generator 22 that generates stimulation pulses for connecting the desired electrodes to the appropriate I/O circuits, thereby facilitating electrode programmability. The pulse generator 22 is controlled by the microcontroller 20 via control signals 24. The IMD 50 includes a sensing circuit 44 selectively coupled to one or more electrodes that perform sensing operations through the switch 26 to detect cardiac activity. The sensing circuit 44 may include dedicated sense amplifiers, multiplexed amplifiers, or shared amplifiers. The sensing circuit 44 may operate in a unipolar sensing configuration or a bipolar sensing configuration. The output of the sensing circuit 44 is connected to the microcontroller 20 which, in turn, triggers, or inhibits the pulse generator 22 in response to the absence or presence of cardiac activity. The sensing circuit 44 receives a control signal 46 from the microcontroller 20 for purposes of controlling the gain, threshold, polarization, and timing of any blocking circuitry (not shown) coupled to the sensing circuit.

The IMD 50 further includes an analog-to-digital A/D data acquisition system (DAS) 84 coupled to one or more electrodes via the switch 26 to sample cardiac signals across any pair of desired electrodes. The A/D DAS 84 is controlled by a control signal 86 from the microcontroller 20.

The IMD 50 is communicatively connected to an external device 90. The external device 90 may communicate with a telemetry circuit 64 of the IMD 50 through a communication link 66. The external device 90 facilitates access by physicians to patient data as well as permitting the physician to review real-time cardiac signals while collected by the IMD 50.

The microcontroller 20 is coupled to a memory 88 by a suitable data/address bus 62. The memory 88 stores the programmable operating parameters used by the microcontroller 20 and/or data associated with the detection and determination of arrhythmias.

The IMD 50 may further include one or more physiologic sensors 70 adjust pacing stimulation rates, detect changes in cardiac output, changes in the physiological condition of the heart, and/or diurnal changes in activity (e.g., detecting sleep and wake states). Examples of physiological sensors 70 might include sensors that, for example, sense respiration rate, pH of blood, ventricular gradient, activity, body movement, position/posture, minute ventilation (MV), and/or the like.

The battery 72 provides operating power to all of the components in the IMD 50. The battery 72 is capable of operating at low current drains for long periods of time, and is capable of providing a high-current pulses (for capacitor charging) when the patient requires a shock pulse (e.g., in excess of 2 A, at voltages above 2 V, for periods of 10 seconds or more).

The IMD 50 further includes an impedance measuring circuit 74, which can be used for many things, including sensing respiration phase. The IMD 50 is further equipped with a communication modem (modulator/demodulator) 40 to enable wireless communication with the external device 90 and/or other external devices.

The IMD 50 includes a shocking circuit 80 controlled by control signals 82 generated by the microcontroller 20. The shocking circuit 80 generates shocking pulses of low (e.g., up to 0.5 joules), moderate (e.g., 0.5-10 joules), or high energy (e.g., 11 to 40 joules), as controlled by the microcontroller 20.

The microcontroller 20 may include other dedicated circuitry and/or firmware/software components, such as a timing control (module) 32 and a morphology detector (module) 36. The timing control 32 is used to control various timing parameters, such as stimulation pulses (e.g., pacing rate, atria-ventricular (AV) delay, atrial interconduction (A-A) delay, ventricular interconduction (V-V) delay, etc.) as well as to keep track of the timing of RR-intervals, refractory periods, blanking intervals, noise detection windows, evoked response windows, alert intervals, marker channel timing, and the like. The morphology detector 36 is configured to review and analyze one or more features of the morphology of cardiac activity signals, such as the morphology of detected R waves to determine whether to include or exclude one or more beats from further analysis.

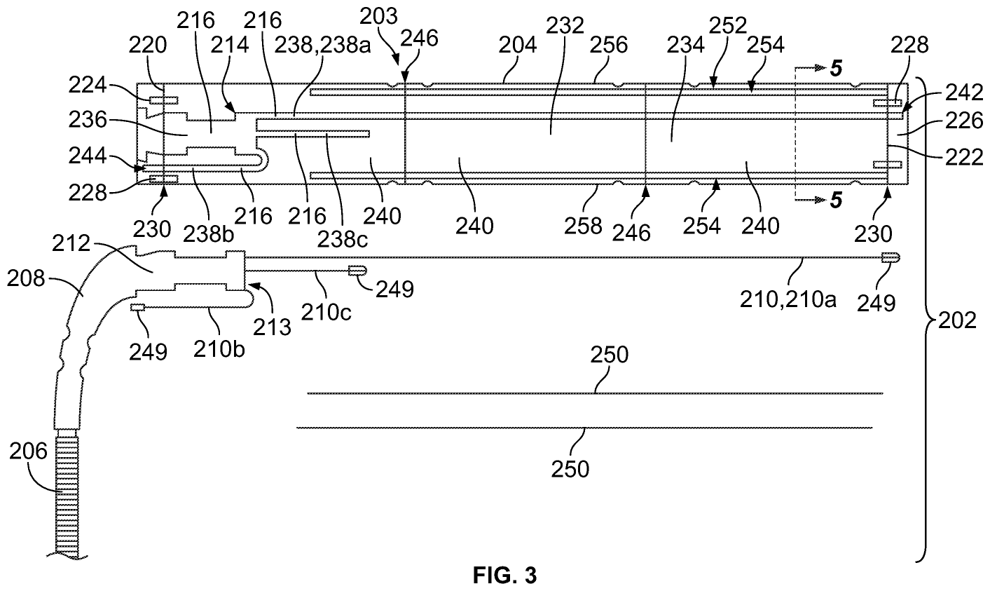

FIG. 3 is an exploded view of a portion of a lead 202 of an IMD according to an embodiment. The lead 202 may be the lead 120 shown in FIG. 1. The lead 202 includes at least one shocking electrode. In the illustrated embodiment, the lead 202 has an electrode module 203 that includes a first (e.g., primary) shocking electrode 204. The lead 202 optionally also includes a second (e.g., secondary) shocking electrode 206. Only a portion of the secondary shocking electrode 206 is shown in FIG. 3. The primary shocking electrode 204 is disposed distal of the secondary shocking electrode 206 (e.g., the shocking electrodes 204, 206 may be referred to as distal and proximal shocking electrodes, respectively).

A segment of the lead 202 extends between the secondary shocking electrode 206 and the electrode module 203. The segment is referred to herein as a cable assembly 208. The cable assembly 208 includes multiple electrical cables 210 collectively surrounded by a tubular member 212. The cable assembly 208 may represent a portion of a lead body, such as the lead body 121 at the gap segment 131 in FIG. 1. For example, the cable assembly 208 may electrically and mechanically connect the lead body to the pulse generator 105 (shown in FIG. 1). The pulse generator 105 may power the shocking electrode 204, via the lead body and the cable assembly 208, to deliver high-voltage shocks for defibrillation therapy. The cable assembly 208 optionally may include one or more components that are not present along at least some other segments of the lead body. For example, the tubular member 212 may be a sleeve that is not present along at least some other segments of the lead body. The sleeve may be a suture sleeve, a strain relief sleeve, and/or the like. The tubular member 212 may be composed of an electrically insulative (e.g., dielectric) material. The insulative material may be or include silicone rubber, polyurethane, and/or the like. The tubular member 212 may form an elbow or curve to change the orientation of the lead 202. The electrical cables 210 may protrude from a distal end 213 of the tubular member 212.

The electrode module 203 is shown separated from the cable assembly 208 in the exploded view of FIG. 3. During assembly of the lead 202, a portion of the cable assembly 208 is loaded into a set 214 of grooves 216 defined along a side of the electrode module 203. The cable assembly 208 is placed into the grooves 216 in a side-loading direction, such as by dropping the cable assembly 208 into the grooves 216 from above. Then, electrical connections may be established between the electrical cables 210 of the cable assembly 208 and different corresponding components of the electrode module 203. The cable assembly 208 may be mechanically secured to the electrode module 203 via covering the cable assembly 208 with cover plates, encasing the cable assembly 208 with an insulative overmold material, applying an adhesive, or the like.

This process for producing the lead 202 may have several advantages over conventional processes. For example, depositing the cable assembly 208 into defined grooves 216 that are open along an elongated surface of the electrode module 203 may be easier and faster than routing the electrical cables 210 along small, closed channels through an end of the electrode module 203. The open grooves 216 may enable better visibility for inspection during the manufacturing process. The open grooves 216 may also provide better access to the electrical cables 210 within the electrode module 203 for establishing electrical connections and/or mechanical connections between the components. For example, rather than forming holes through a thickness of an electrode to spot-weld an electrical cable across the thickness, welding operations can be directly performed along the surface of the electrode that defines the grooves 216 without forming holes or making other accommodations. Furthermore, the set 214 of grooves 216 may be designed to provide inherent strain relief by interlocking with the tubular member 212 of the cable assembly 208 when the cable assembly 208 is loaded into the grooves 216.

The primary shocking electrode 204 (referred to hereafter simply as shocking electrode 204) extends from a proximal end 220 of the electrode 204 to a distal end 222 of the electrode 204. In an embodiment, the electrode module 203 includes a proximal sensing electrode 224 and a distal sensing electrode 226 that bookend the shocking electrode 204. The sensing electrodes 224, 226 may represent the sensing electrode(s) 148 described in FIG. 1, such that the sensing electrodes sense cardiac signals. The sensing electrodes 224, 226 optionally may be used to deliver electrical stimulation in the form of pacing pulses, which have a lower voltage than the high-voltage shocks delivered by the shocking electrode 204. The proximal sensing electrode 224 is disposed beyond the proximal end 220 of the shocking electrode 204, and the distal sensing electrode 226 is disposed beyond the distal end 222. The shocking electrode 204 may be mechanically connected to the sensing electrodes 224. In an example, dowel pins 228 (shown in phantom) may be installed into ends of the shocking electrode 204 and the sensing electrodes 224, 226. The dowel pins 228 extend across the corresponding interfaces 230 between the components to secure the sensing electrodes 224, 226 to the shocking electrode 204. In an alternative embodiment, rather than dowel pins, the electrodes 204, 224, 226 may be affixed together via an adhesive, an overmold material, or another type of mechanical fastener.

In an embodiment, the set 214 of grooves 216 are defined in at least a base structure 232 of the shocking electrode 204. The base structure 232 may represent a structural frame (e.g., core) of the shocking electrode 204. The base structure 232 may extend from the proximal end 220 to the distal end 222 of the shocking electrode 204. The base structure 232 has a first side 234, which is the illustrated side in FIG. 3. The base structure 232 may have an oblong cross-sectional shape, as shown in FIG. 5. The first side 234 represents one of the perimeter sides of the oblong cross-sectional shape. The first side 234 may extend the full length of the base structure 232 between the proximal and distal ends 220, 222. The set 214 of grooves 216 is defined along the first side 234. For example, the grooves 216 may be recesses, troughs, indentations, depressions, or the like that are open along the surface of the first side 234. As opposed to longitudinal channels that are disposed entirely within a thickness of a structure, the grooves 216 extend from the first side 234 partially into the thickness of the base structure 232. The full length of the grooves 216 shown in FIG. 3 are visible and exposed to an observer, such as an operator that assembles the lead 202.

The grooves 216 in the set 214 may be sized, shaped, and positioned to receive the cable assembly 208 therein. Different grooves 216 in the set 214 may be designed to receive different corresponding components of the cable assembly 208. For example, the grooves 216 may include a recess 236 sized, shaped, and positioned to receive at least a portion of the tubular member 212 of the cable assembly 208. The grooves 216 may include slots 238 that extend from the recess 236. Each of the slots 238 may be sized, shaped, and positioned to receive a different corresponding electrical cable 210 of the cable assembly 208. The slots 238 may be narrower than the recess 236. The term “sized” as used herein is not limited to the act of manufacturing, but rather refers to a dimension of a first component, similar to length, width, volume, etc., where the dimension may be selected based on a dimension of at least a second component. The grooves 216 being sized to receive the cable assembly 208 is not a method operation, but rather a characteristic or property of the base structure 232 in which the grooves 216 are sufficiently wide, deep, long, and/or the like to accommodate at least a portion of the cable assembly 208 therein.

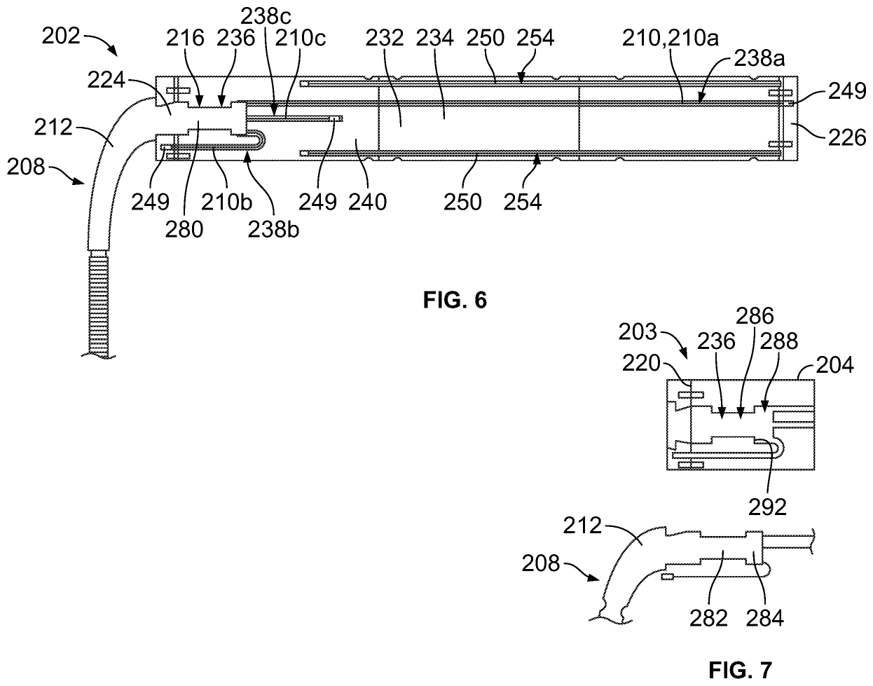

In an embodiment, the set 214 of grooves 216 continues onto the proximal and distal sensing electrodes 224, 226. For example, the distal sensing electrode 226 defines a slot 242 that aligns with, and extends from, a first slot 238a of the shocking electrode 204. The slots 238a, 242 may receive a first electrical cable 210a of the cable assembly 208 to electrically connect to the distal sensing electrode 226. The proximal sensing electrode 224 may define a slot 244 that aligns with, and extends from, a second slot 238b of the shocking electrode 204. The slots 238b, 244 may receive a second electrical cable 210b of the cable assembly 208 to electrically connect to the proximal sensing electrode 224. The grooves 216 may include a third slot 238c designed to receive a third electrical cable 210c. The third electrical cable 210c may electrically connect to the shocking electrode 204, when the lead 202 is assembled as shown in FIG. 6.

In a first embodiment, the base structure 232 is modular and defined by multiple, discrete brick segments 240. The brick segments 240 are mechanically connected to one another in a line. The first side 234 of the base structure 232 extends along the series of brick segments 240. The set 214 of grooves 216 continuously extends along at least two of the brick segments 240. In the illustrated example, the recess 236 is disposed on the most proximal brick segment 240, and the first slot 238a extends along all the brick segments 240 to the distal end 222 of the shocking electrode 204.

The brick segments 240 may be chicklets, chips, pieces, chunks, tablets, or the like. The brick segments 240 may be replicas or copies of one another, such that the brick segments 240 may have the same shapes, dimensions, and features. The term “brick segment” refers to how the pieces in combination form the base structure 232 of the shocking electrode 204, without inherently denoting or requiring any specific shape. The brick segments 240 may line up as they are assembled to form the base structure 232.

The length of the shocking electrode 204 may be the determined by the respective lengths of the brick segments 240 and the number of brick segments 240 in the line. There are three brick segments 240 in the illustrated embodiment, but two, four, or more than four brick segments 240 are possible. A benefit of the modular base structure 232 is that the length of the shocking electrode 204 can be customized for a patient by selecting the number of brick segments 240 to include in the line. For example, the shocking electrode 204 may be constructed with more brick segments 240 for implantation within a tall adult, and fewer brick segments 240 for implantation in a juvenile or short adult. Adjacent brick segments 240 are coupled together at joints 246. The shocking electrode 204 has seams at the joints 246. The seams at the joints 246 may promote tissue in-growth to reduce the risk of lead/electrode migration from the implanted location over time.

In an example, each brick segment 240 may have an oblong cross sectional shape, which is shown in FIG. 5. The cross-sectional shape defined by the outer perimeter of the brick segment 240 may be a racetrack, rectangle with curved corners, elliptical, oval, or the like.

In an embodiment, the brick segments 240 are electrically conductive, and are electrically connected to one another across the joints 246. For example, the brick segments 240 in the line may be electrically commoned with one another (e.g., at the same electrical potential). The shocking electrode 204 may receive electrical power (e.g., electric current) from the pulse generator via the third electrical cable 210c, which is also referred to herein as a power cable 210c. The brick segments 240 may emit the electrical power that is received as the high-voltage shocks for defibrillation therapy. A portion of the power cable 210c may be welded, crimped, or otherwise securely electrically connected to at least one of the brick segments 240 to establish an electrically conductive pathway between the pulse generator to the shocking electrode 204.

The brick segments 240 may be composed of one or more electrically conductive materials that are safe for human tissue interaction. The brick segments 240 may include one or more metals, such as titanium, nickel, chromium, cobalt, stainless steel, and/or the like. In an example, the brick segments 240 may be machined, such that the brick segments 240 are initially formed as solid pieces and then a subsequent process is performed to extract material from the solid pieces to define the grooves 216. The subsequent process may be drilling or the like. In another example, the brick segments 240 may be formed by molding (e.g., casting) or another process other than machining.

In embodiments in which the base structure 232 of the shocking electrode 204 is electrically conductive, the electrical cables 210 and sensing electrodes 224, 226 may be electrically insulated from the base structure 232. For example, the electrical cables 210 may be insulated wires, insulated cables, and/or the like. Electrically insulative membranes may be installed at the interfaces 230 between the ends 220, 222 of the shocking electrode 204 and the sensing electrodes 224, 226. The electrical cables 210 may have electrically conductive tips 249 at the distal ends thereof. The electrically conductive tips 249 may be crimp sleeves, exposed sections of wire, and/or the like. The tips 249 may be welded or otherwise secured in direct mechanical contact to the corresponding electrodes 204, 224, 226 to electrically connect the electrical cables 210 to the electrodes 204, 224, 226.

In an embodiment, the brick segments 240 are mechanically connected to each other via one or more support wires 250. The shocking electrode 204 includes two support wires 250 in the illustrated embodiment, but other embodiments may include only one support wire or at least three support wires. The support wires 250 extend across the joints 246 between the brick segments 240. The support wires 250 may extend along each of the brick segments 240. The support wires 250 are affixed to at least some of the brick segments 240 to secure the brick segments 240 to one another in the line. For example, the support wires 250 exert tension to prevent the brick segments 240 from separating at the joints 246. Optionally, the support wires 250 may be affixed to each of the brick segments 240. Alternatively, the support wires 250 may be affixed to a subset, but not all, of the brick segments 240, and tension between the brick segments 240 may hold non-affixed brick segments 240 in place. The support wires 250 may be affixed to the brick segments 240 via welding, crimping, bonding, or the like.

The support wires 250 may be formed of any material that provides sufficient strength to structurally support the shocking electrode 204. In an embodiment, the support wires 250 are electrically conductive. In addition to providing mechanical support and retention, the electrically conductive support wires 250 may provide electrically conductive pathways between the brick segments 240 to electrically connect the brick segments 240. For example, the support wires 250 may be formed of a metal material, such as stainless steel.

In an embodiment, the support wires 250 may be assembled to the brick segments 240 (e.g., the base structure 232) by side-loading the support wires 250 into another (e.g., second) set 252 of one or more grooves 254 defined along the first side 234. The grooves 254 are spaced apart and discrete from the grooves 216 in the first set 214. The grooves 254 are sized, shaped, and positioned to accommodate the support wires 250. In the illustrated embodiment, the brick segments 240 include two grooves 254 corresponding to the two support wires 250. The two grooves 254 are disposed proximal to first and second lateral sides (e.g., edges) 256, 258, respectively, of the base structure 232. During the assembly process, the support wires 250 may be side-loaded (e.g., dropped) into the corresponding grooves 254, similar to the loading of the portion of the cable assembly 208 into the first set 214 of grooves 216. Once loaded, the support wires 250 may be secured within the grooves 254 via crimping the brick segments 240, welding, applying an adhesive, applying an overmold material to encase the support wires 250, and/or the like. Once secured in the grooves 254, the support wires 250 retain the positioning of the brick segments 240 in the line.

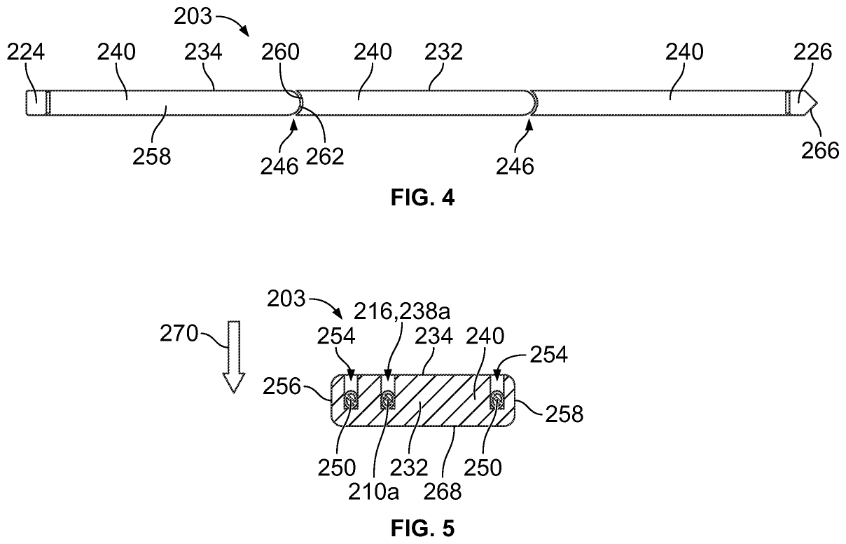

FIG. 4 is a side view of the electrode module 203 of FIG. 3 according to an embodiment. FIG. 4 shows the second lateral side 258 of the base structure 232. In an embodiment, the brick segments 240 are at least partially nested together at the joints 246. For example, at each joint 246, one of the brick segments 240 has a concave mating surface 260 and the other brick segment 240 has a convex mating surface 262 that nests into the concave mating surface 260. At the nested joints 246, the end of one of the brick segments 240 may be at least partially received into the end of the adjacent brick segment 240 such that there is some overlap between the two brick segments 240.

In an embodiment, the brick segments 240 may be pivotable at the nested joints 246 in at least one dimension (e.g., one degree of freedom). In an example, the electrode module 203 can flex in the vertical (e.g., up and down) dimension at the joints 246, causing the electrode module 203 to have a bowed and/or undulating shape in the vertical dimension along its length. The tension of the support wires 250 (shown in FIG. 3) may be sufficiently relaxed to enable at least slight flexibility of the shocking electrode 204 at the joints 246. The flexible characteristic of the electrode module 203 may beneficially allow the electrode module 203 to follow a contour of the patient's body structure upon implant, such as along the contour of the sternum. Furthermore, the flexible electrode module 203 may adapt to the contour of the patient's body even as the patient moves within a normal range of movement, to avoid causing pain or discomfort, and to avoid excessive protrusion underneath the skin that could contribute to body dysmorphia issues. Optionally, the electrode module 203 may also flex in at least one other dimension, such as the lateral (e.g., side-to-side) dimension.

Optionally, the distal end of the electrode module 203 may be tapered (e.g., wedge-shaped) to enable blunt dissection of and/or tunneling through patient tissue during implant. For example, the distal sensing electrode 226 may be tapered with a wedge shape 266 to reduce the input force required during implantation and reduce the risk of tissue damage due to snagging, relative to more devices having more blunt distal ends.

FIG. 5 is a cross-sectional view of the electrode module 203 according to an embodiment. The cross-section is taken along line 5-5 in FIG. 3, and extends through one of the brick segments 240 that define the base structure 232. In an embodiment, the brick segment 240 (e.g., base structure 232) has an oblong cross-sectional shape. The oblong cross-sectional shape includes the first side 234 (along the upper perimeter in FIG. 5), a second side 268 opposite the first side 234 (along the lower perimeter in FIG. 5), and the first and second lateral sides 256, 258. The lateral sides 256, 258 are disposed opposite each other and extend between the first and second sides 234, 268. The sides 234, 268 may be larger than the lateral sides 256, 258. For clarity, the sides 234, 268 are referred to as first and second broad sides 234, 268 herein, and the lateral sides 256, 258 are referred to as first and second narrow sides 256, 258. In the illustrated embodiment, the oblong cross-sectional shape is a rectangle with curved/rounded corners and planar sides 234, 256, 258, 268. In other embodiments, the brick segments 240 may have racetrack shapes, elliptical shapes, oval shapes, and/or the like.

In an embodiment, the grooves 216, 254 are defined along the first broad side 234 and extend into the interior (e.g., thickness) of the brick segments 240 (e.g., base structure 232) towards the second broad side 268. The grooves 216, 254 bottom out within the interior of the brick segments 240, such that the grooves 216, 254 are only open along the first broad side 234, not the second broad side 268.

In an embodiment, during assembly, the portion of the cable assembly 208 and the support wires 250 may be placed into the corresponding grooves 216, 254 in a side-loading direction 270. The side-loading direction 270 is oriented to face from the first broad side 234 towards the second broad side 268. This loading direction is in contrast with some conventional lead assembly techniques which feed electrical cables into one or more channels of a shocking electrode in a longitudinal direction (e.g., into and/or out of the page in the orientation shown in FIG. 5). In FIG. 5, the cross-section line extends through both of the grooves 254 that receive the electrical wires 250 and the first slot 238a that receives the first electrical cable 210a. The cable 210a and the electrical wires 250 may be simply dropped and/or pressed into the corresponding grooves 216, 254, from along the first broad side 234, to achieve the loaded positions that are shown. The grooves 216, 254 along other portions of the electrode module 203 may similarly be formed to extend from the first broad side 234 towards the second broad side 268.

FIG. 6 is a plan view showing the lead 202 of FIG. 3 in a partially assembled state. During the assembly process, a portion of the cable assembly 208 is placed into the grooves 216. The portion within the grooves 216 may include a distal segment 280 of the tubular member 212 and the segments of the electrical cables 210 that project from the distal segment 280 of the tubular member 212. For example, the distal segment 280 is disposed within the recess 236. The first electrical cable 210a is disposed within the first slot 238a, which extends to the distal sensing electrode 226. The second electrical cable 210b is disposed within the second slot 238b, which extends to the proximal sensing electrode 224. The third electrical cable 210c is disposed within the third slot 238c, which ends at the most proximal brick segment 240. The assembly process includes electrically connecting the electrical cables 210 to the corresponding grooves 216. In an embodiment, the distal tip 249 of the first electrical cable 210a may be welded to the distal sensing electrode 226, the distal tip 249 of the second electrical cable 210b may be welded to the proximal sensing electrode 224, and the distal tip 249 of the third electrical cable 210c may be welded to the most proximal brick segment 240. In other embodiments, the distal tips 249 may be crimped to the corresponding components, bonded, or the like.

During assembly, the support wires 250 are placed into the grooves 254 in the brick segments 240 and are secured to the brick segments 240. The support wires 250 may be crimped, bonded, welded, or the like. In the partially assembled state, the cable assembly 208 and/or the support wires 250 may be exposed (e.g., uncovered) along the first broad side 234 of the base structure 232.

FIG. 7 is an enlarged view of a portion of the lead 202 showing the recess 236 in the electrode module 203 and the tubular member 212 of the cable assembly 208 according to an embodiment. In an embodiment, the distal segment 280 of the tubular member 212 has features that interlock with complementary surfaces of the recess 236. Upon loading the distal segment 280 into the recess 236, abutting surfaces provide strain relief by resisting axial movement of the tubular member 212 relative to the electrode module 203.

In an embodiment, the tubular member 212 includes a neck 282 and a flange 284 that has a greater diameter than the neck 282. The flange 284 is distal of the neck 282. The recess 236 includes a narrow segment 286 and a broad segment 288. The narrow segment 286 is proximal of the broad segment 288 (e.g., disposed between the proximal end 220 of the base structure 232 and the broad segment 288). The narrow segment 286 is sized and shaped to receive the neck 282 of the tubular member 212, and the broad segment 288 is sized and shaped to receive the flange 284 of the tubular member 212. The flange 284 has a greater width (e.g., diameter) than the narrow segment 286 to axially secure the cable assembly 208 to the shocking electrode 204. For example, tension on the cable assembly 208 in the proximal direction relative to the electrode module 203 would cause the flange 284 to abut against a shoulder 292 of the recess 236 between the narrow and broad segments 286, 288, which resists the pull-out force.

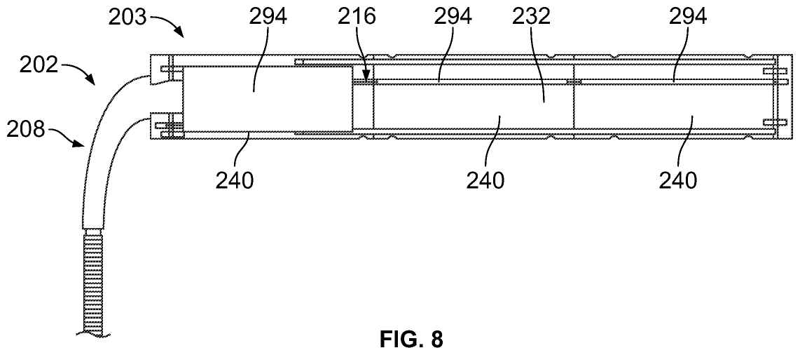

FIG. 8 is a plan view showing the lead 202 of FIG. 3 in a fully assembled state according to an embodiment. In an embodiment, the lead 202 may include one or more cover plates 294 that cover the portion of the cable assembly 208 that is within the grooves 216. Once covered, the cable assembly 208 may be disposed between the cover plate(s) 294 and the second broad side 268 (shown in FIG. 5) of the base structure 232. In FIG. 8, different cover plates 294 are mounted to each of the different brick segments 240. As such, the cover plates 294 may not interfere with the flexibility of the electrode module 203. The cover plates 294 optionally may be thin sheets of a metal material that is safe for human tissue interaction. The cover plates 294 may be welded to the brick segments 240, bonded to the brick segments 240 and/or cable assembly 208, or the like.

The lead 202 may be a subcutaneous lead. When implanted, the electrode module 203 may be oriented such that the first broad side 234 faces away from the patient's heart, towards the underside of the patient's skin. The second broad side 268 may face towards the patient's heart and rib cage.

In an alternative embodiment, the lead 202 lacks the cover plates 294. For example, the lead 202 in the state shown in FIG. 6 may represent the fully assembled state. In another embodiment, the electrode module 203 (e.g., including the shocking electrode 204) includes an overmold material that is applied on the first broad side 234 of the base structure 232 (e.g., the brick segments 240) and the portion of the cable assembly 208 disposed in the grooves 216.

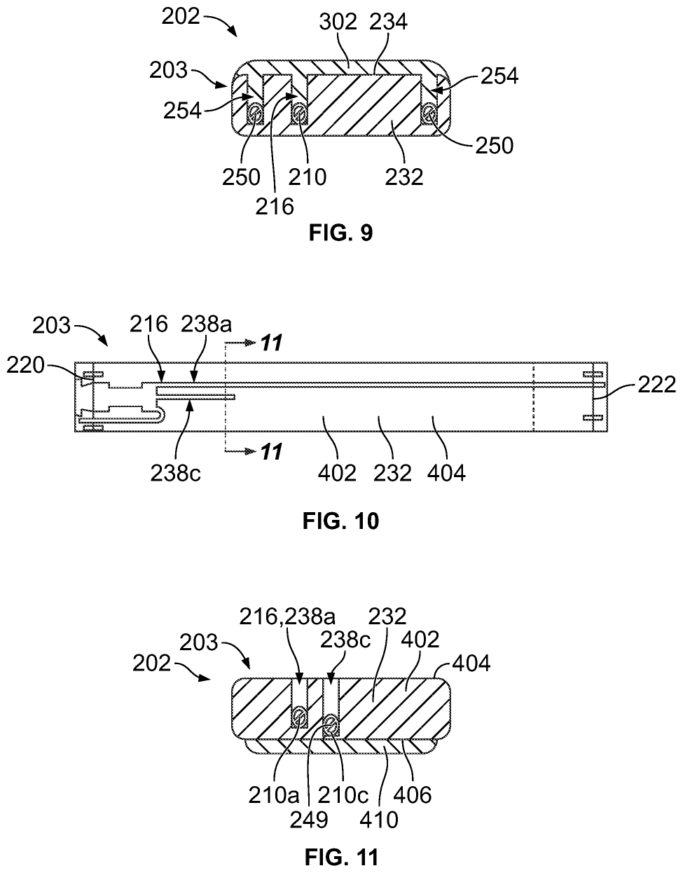

FIG. 9 is a cross-sectional view of the lead 202 including an overmold material 302 on the electrode module 203 according to an embodiment. The cross-section may be taken along line 5-5 in FIG. 3. The overmold material 302 may be applied in a flowable state. The overmold material 302 may flow along and conform to the contours of the cable assembly 208 (shown in FIG. 6) and the base structure 232. In the illustrated embodiment, the overmold material 302 is disposed on the first broad side 234 and enters the grooves 216, 254. When the overmold material 302 hardens and solidifies, the cable assembly 208 may be encased between the base structure 232 and the overmold material 302. For example, the electrical cables 210 and the support wires 250 may be encased between the base structure 232 and the overmold material 302. In an example, the overmold material 302 may be an electrically insulative material. The insulative material may be or include silicone rubber, polyurethane, and/or the like. In another embodiment, the overmold material 302 may be electrically conductive.

The modular shocking electrode 204 shown in FIGS. 3 through 9 can be made at variable lengths to create longer/shorter shocking electrodes as needed while still having increased flexibility compared to a solid piece of metal. The added surface area of the oblong electrodes help decrease shocking impedance, which in turn reduces the required device capacitance requirements. The reduced device capacitance requirements may permit reducing the size of the pulse generator device.

FIG. 10 is a plan view of the electrode module 203 according to an alternative embodiment. FIG. 11 is a cross-sectional view of the lead 202 including the electrode module 203 of FIG. 10. The cross-section is taken along line 11-11 in FIG. 10. In the illustrated embodiment, the base structure 232 of the shocking electrode 204 is a single, unitary (e.g., monolithic) body 402. The body 402 extends from the proximal end 220 to the distal end 222 of the shocking electrode 204. In an embodiment, the body 402 is formed of an electrically insulative material, such that the body 402 is referred to as an insulative body 402. Because the insulative body 402 is unitary, there are no joints or support wires along the length. The body 402 defines the grooves 216 that receive the cable assembly 208. For example, the body 402 has an oblong cross-sectional shape with a first side 404 (referred to as first broad side) and a second side 406 (referred to as second broad side) opposite the first broad side 404.

In an embodiment, the shocking electrode 204 includes an electrically conductive layer 410 disposed along the second broad side 406. The electrically conductive layer 410 may extend for at least a majority of the length of the shocking electrode 204 between the ends 220, 222. The electrically conductive layer 410 may deliver the high-voltage shocks. For example, the power cable 210c of the cable assembly 208 may be electrically connected to the electrically conductive layer 410 to establish a conductive pathway from the pulse generator of the IMD to the electrically conductive layer 410. For example, a hole may be formed at the distal end of the slot 238c that enables the distal tip 249 of the cable 210c to penetrate through the base structure 232 and contact the electrically conductive layer 410, as shown in FIG. 11. The distal tip 249 may be welded or otherwise affixed to the electrically conductive layer 410. In an example, the electrically conductive layer 410 may include one or more metal plates affixed to the second broad side 406 of the insulative body 402. In an alternative embodiment, a metallic wire coil may be wrapped around the insulative body 402 instead of securing metal plates to the second broad side 406.

The electrode module 203 in the embodiments shown in FIGS. 3 through 11 include the shocking electrode 204 between two sensing electrodes 224, 226. In an alternative embodiment, the electrode module 203 may lack one or both of the sensing electrodes 224, 226. For example, the electrode module 203 may only include the shocking electrode 204, may include only the shocking electrode 204 and the distal sensing electrode 226, or may include only the shocking electrode 204 and the proximal sensing electrode 224. Furthermore, although the electrode module 203 is described with reference to the primary (e.g., distal) shocking electrode 204, the embodiments may be applied to the secondary (e.g., proximal) shocking electrode 206. For example, in a first embodiment, only the distal shocking electrode 204 defines the grooves 216. In a second embodiment, both the distal and proximal electrodes 204, 206 may define grooves along respective first sides to receive different portions of the cable assembly. In a third embodiment, only the proximal shocking electrode 206 defines grooves along the side to receive a portion of the cable assembly.

Optionally, the base structure 232 may include a trough defined along the first side 234, 404. The trough may be a groove that is spaced apart from the set 214 of grooves 216 and the grooves 254. The trough may be sized and shaped to receive an elongated implant tool therein through the proximal end 220 of the shocking electrode 204. The implant tool may include a linear rod that extends through the trough. The implant tool within the trough may be used to manipulate the lead 202 during the implant procedure, such as to guide and advance the electrode module 203 of the lead 202. Optionally, the trough may be a closed lumen, rather than an open-faced groove.

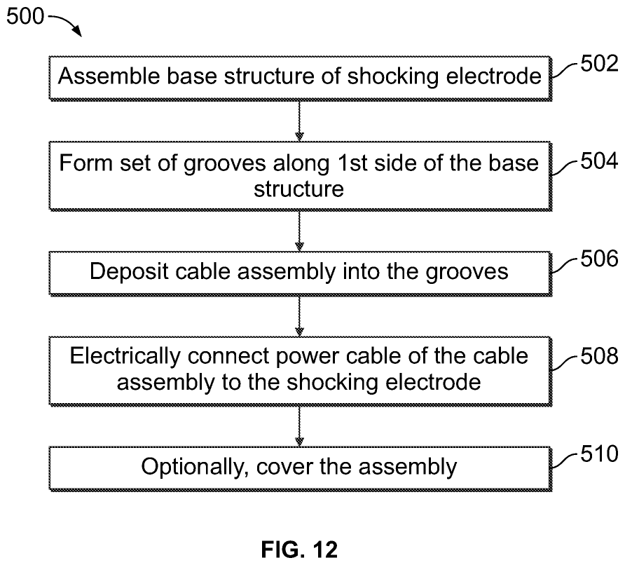

FIG. 12 is a flow chart 500 of a method for producing a lead for an implantable medical device (IMD) according to an embodiment. The lead that is produced according to the method in FIG. 12 may be the lead 202 according to the embodiments described with reference to FIGS. 3 through 11. The method may include additional steps than shown in FIG. 12, fewer steps than shown in FIG. 12, and/or different steps than shown in FIG. 12.

At step 502, a base structure 232 of a shocking electrode 204 is assembled. The shocking electrode 204 is configured to deliver high-voltage shocks for defibrillation therapy. In a first embodiment, the base structure 232 may be assembled by mechanically connecting a plurality of discrete brick segments 240 together in a line. The brick segments 240 may be electrically conductive and electrically connected to one another. The brick segments may be secured to one another via support wires 250. The base structure 232 may be assembled by forming grooves 254 along the brick segments 240, and then side-loading the support wires 250 into the grooves 254 and securing the support wires 250 within the grooves 254. In a second embodiment, the base structure 232 may be assembled by obtaining an insulative body 402 that extends the length of the shocking electrode 204, and applying an electrically conductive layer 410 along at least a second side 406 of the insulative body 402. The electrically conductive layer 410 may be one or more metal plates.

At step 504, a set 214 of grooves 216 is formed along a first side 234, 404 of the base structure 232 of the shocking electrode 204. The base structure 232 has an oblong cross-section shape with a second side 268, 406 that is opposite the first side 234, 404. In the embodiment in which the base structure 232 includes the brick segments 240, the grooves 216 are formed along the first side 234 of at least two of the brick segments 240. In the embodiment in which the base structure 232 has the insulative body 402, the grooves 216 are formed along the first side 404 that is opposite the electrically conductive layer 410.

At step 506, a portion of a cable assembly 208 is deposited into the grooves 216 of the set 214 in a side-loading direction 270. The cable assembly 208 may include multiple cables 210 and a tubular member 212 that collectively surrounds the cables 210. The set 214 of grooves 216 may include a recess 236 and multiple slots 238 extending from the recess 236. Depositing the portion of the cable assembly 208 into the grooves 216 may include placing the tubular member 212 into the recess 236 and placing the cables 210 into different corresponding slots 238 of the set 214 of grooves 216.

At step 508, a power cable 210c of the cables 210 is electrically connected to the shocking electrode 204 via either (i) one of the brick segments 240, or (ii) the electrically conductive layer 410. For example, a distal tip 249 of the power cable 210c may be welded to the brick segment 240 or the electrically conductive layer 410, while the power cable 210c is within the corresponding groove 216 (e.g., slot 238c). The power cable 210c may be electrically connected to a pulse generator.

At step 510, as an optional step, the portion of the cable assembly 208 within the grooves 216 may be covered. For example, one or more cover plates 294 may be applied over the cable assembly 208 such that the cable assembly 208 is disposed between the one or more cover plates 294 and the second side 268, 406 of the base structure 232. In another example, an overmold material 302 may be applied on the first side 234, 404 of the base structure 232 and the cable assembly 208 such that the overmold material 302 conforms to a contour of the cable assembly 208 and encases the cable assembly 208 between the base structure 232 and the overmold material 302.

The method may further include implanting the lead 202 such that the shocking electrode 204 is disposed in a subcutaneous location within the patient. The first side 234 may be oriented to face the underside of the skin (e.g., away from the heart and ribcage).

### Closing

It should be clearly understood that the various arrangements and processes broadly described and illustrated with respect to the Figures, and/or one or more individual components or elements of such arrangements and/or one or more process operations associated of such processes, can be employed independently from or together with one or more other components, elements and/or process operations described and illustrated herein. Accordingly, while various arrangements and processes are broadly contemplated, described and illustrated herein, it should be understood that they are provided merely in illustrative and non-restrictive fashion, and furthermore can be regarded as but mere examples of possible working environments in which one or more arrangements or processes may function or operate.

As will be appreciated by one skilled in the art, various aspects may be embodied as a system, method or computer (device) program product. Accordingly, aspects may take the form of an entirely hardware embodiment or an embodiment including hardware and software that may all generally be referred to herein as a “circuit,” “module” or “system.” Furthermore, aspects may take the form of a computer (device) program product embodied in one or more computer (device) readable storage media having computer (device) readable program code embodied thereon.

Any combination of at least one non-signal computer (device) readable medium may be utilized. The non-signal medium may be a storage medium. A storage medium may be, for example, an electronic, magnetic, optical, electromagnetic, infrared, or semiconductor system, apparatus, or device, or any suitable combination of the foregoing. More specific examples of a storage medium would include the following: a portable computer diskette, a hard disk, a random access memory (RAM), a dynamic random access memory (DRAM), a read-only memory (ROM), an erasable programmable read-only memory (EPROM or Flash memory), a portable compact disc read-only memory (CD-ROM), an optical storage device, a magnetic storage device, or any suitable combination of the foregoing.

Program code for carrying out operations may be written in any combination of one or more programming languages. The program code may execute entirely on a single device, partly on a single device, as a stand-alone software package, partly on single device and partly on another device, or entirely on the other device. In some cases, the devices may be connected through any type of network, including a local area network (LAN) or a wide area network (WAN), or the connection may be made through other devices (for example, through the Internet using an Internet Service Provider) or through a hard wire connection, such as over a USB connection. For example, a server having a first processor, a network interface, and a storage device for storing code may store the program code for carrying out the operations and provide this code through its network interface via a network to a second device having a second processor for execution of the code on the second device.

Aspects are described herein with reference to the figures, which illustrate example methods, devices and program products according to various example embodiments. The program instructions may be provided to a processor of a general-purpose computer, special purpose computer, or other programmable data processing device or information handling device to produce a machine, such that the instructions, which execute via a processor of the device implement the functions/acts specified. The program instructions may also be stored in a device readable medium that can direct a device to function in a particular manner, such that the instructions stored in the device readable medium produce an article of manufacture including instructions which implement the function/act specified. The program instructions may also be loaded onto a device to cause a series of operational steps to be performed on the device to produce a device implemented process such that the instructions which execute on the device provide processes for implementing the functions/acts specified.

The units/modules/applications herein may include any processor-based or microprocessor-based system including systems using microcontrollers, reduced instruction set computers (RISC), application specific integrated circuits (ASICs), field-programmable gate arrays (FPGAs), logic circuits, and any other circuit or processor capable of executing the functions described herein. Additionally, or alternatively, the modules/controllers herein may represent circuit modules that may be implemented as hardware with associated instructions (for example, software stored on a tangible and non-transitory computer readable storage medium, such as a computer hard drive, ROM, RAM, or the like) that perform the operations described herein. The above examples are exemplary only, and are thus not intended to limit in any way the definition and/or meaning of the term “controller.” The units/modules/applications herein may execute a set of instructions that are stored in one or more storage elements, in order to process data. The storage elements may also store data or other information as desired or needed. The storage element may be in the form of an information source or a physical memory element within the modules/controllers herein. The set of instructions may include various commands that instruct the modules/applications herein to perform specific operations such as the methods and processes of the various embodiments of the subject matter described herein. The set of instructions may be in the form of a software program. The software may be in various forms such as system software or application software. Further, the software may be in the form of a collection of separate programs or modules, a program module within a larger program or a portion of a program module. The software also may include modular programming in the form of object-oriented programming. The processing of input data by the processing machine may be in response to user commands, or in response to results of previous processing, or in response to a request made by another processing machine.

It is to be understood that the subject matter described herein is not limited in its application to the details of construction and the arrangement of components set forth in the description herein or illustrated in the drawings hereof. The subject matter described herein is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

It is to be understood that the above description is intended to be illustrative, and not restrictive. For example, the above-described embodiments (and/or aspects thereof) may be used in combination with each other. In addition, many modifications may be made to adapt a particular situation or material to the teachings herein without departing from its scope. While the dimensions, types of materials and coatings described herein are intended to define various parameters, they are by no means limiting and are illustrative in nature. Many other embodiments will be apparent to those of skill in the art upon reviewing the above description. The scope of the embodiments should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects or order of execution on their acts.

What is claimed is:

1. A lead of an implantable medical device (IMD), the lead comprising:
a shocking electrode configured to deliver high-voltage shocks for defibrillation therapy, the shocking electrode including a base structure that has an oblong cross-sectional shape with a first side and a second side that is opposite the first side,
wherein the base structure has a set of grooves defined along the first side, the grooves in the set configured to receive a cable assembly that is placed into the grooves in a side-loading direction.

2. The lead of claim 1, further comprising the cable assembly, the cable assembly comprising multiple cables and a tubular member that collectively surrounds the cables, wherein the set of grooves includes a recess configured to accommodate the tubular member and multiple slots extending from the recess and configured to accommodate the cables.

3. The lead of claim 2, wherein the tubular member includes a neck and a flange that has a greater diameter than the neck, the recess in the set of grooves including a narrow segment and a broad segment, with the narrow segment disposed between a proximal end of the base structure and the broad segment, wherein the narrow segment is configured to receive the neck of the tubular member and the broad segment is configured to receive the flange of the tubular member, and the flange has a greater diameter than the narrow segment to axially secure the cable assembly to the shocking electrode.

4. The lead of claim 1, wherein the base structure is defined by multiple brick segments that are discrete and mechanically connected to one another in a line, wherein the set of grooves is defined along the first side of at least two of the brick segments.

5. The lead of claim 4, wherein the brick segments are electrically conductive and electrically connected to one another, wherein a power cable of the cable assembly is welded to one of the brick segments to establish a conductive pathway from a pulse generator of the IMD to the shocking electrode.

6. The lead of claim 4, wherein the set of grooves is a first set of grooves and the base structure further includes a second set of one or more grooves defined along the first side and spaced apart from the first set of grooves, wherein the shocking electrode further comprises one or more support wires disposed within the second set of the one or more grooves and affixed to the brick segments to secure the brick segments to one another in the line.

7. The lead of claim 1, wherein the base structure is an insulative body that extends from a proximal end of the shocking electrode to a distal end of the shocking electrode, wherein the shocking electrode includes an electrically conductive layer disposed along the second side of the insulative body, the electrically conductive layer configured to be electrically connected to a power cable of the cable assembly to establish a conductive pathway from a pulse generator of the IMD to the electrically conductive layer to deliver the high-voltage shocks for the defibrillation therapy.

8. The lead of claim 7, wherein the electrically conductive layer includes one or more metal plates affixed to the second side of the insulative body.

9. The lead of claim 1, wherein the shocking electrode includes one or more cover plates that cover a portion of the cable assembly that is within the set of grooves, such that the portion of the cable assembly is disposed between the one or more cover plates and the second side of the base structure.

10. The lead of claim 1, wherein the shocking electrode includes an overmold material on the first side of the base structure and a portion of the cable assembly that is within the set of grooves, wherein the overmold material conforms to a contour of the portion of the cable assembly and encases the portion of the cable assembly between the base structure and the overmold material.

11. A method of producing a lead for an implantable medical device (IMD), the method comprising:
forming a set of grooves along a first side of a base structure of a shocking electrode configured to deliver high-voltage shocks for defibrillation therapy, the base structure having an oblong cross-section shape with a second side that is opposite the first side; and
depositing a portion of a cable assembly into the grooves of the set in a side-loading direction.

12. The method of claim 11, wherein the cable assembly includes multiple cables and a tubular member that collectively surrounds the cables, and the set of grooves includes a recess and multiple slots extending from the recess, wherein depositing the portion of the cable assembly into the grooves includes placing the tubular member into the recess and placing the cables into different corresponding slots of the set of grooves.

13. The method of claim 11, further comprising assembling the base structure by mechanically connecting a plurality of discrete brick segments together in a line, wherein the set of grooves is formed along the first side of at least two of the brick segments.

14. The method of claim 13, wherein the brick segments are electrically conductive and electrically connected to one another, and the method further comprises welding a power cable of the cable assembly to one of the brick segments to establish a conductive pathway from a pulse generator of the IMD to the shocking electrode.

15. The method of claim 13, wherein the set of grooves is a first set of grooves and the base structure further includes a second set of one or more grooves defined along the first side and spaced apart from the first set of grooves, wherein the method further comprises depositing one or more support wires in the side-loading direction into the second set of the one or more grooves and affixing the one or more support wires to the brick segments to secure the brick segments to one another in the line.

16. The method of claim 11, wherein the base structure is an insulative body that extends from a proximal end of the shocking electrode to a distal end of the shocking electrode, and the method further comprises:
applying an electrically conductive layer along the second side of the insulative body; and
affixing a power cable of the cable assembly to the electrically conductive layer to establish a conductive pathway from a pulse generator of the IMD to the electrically conductive layer to deliver the high-voltage shocks for the defibrillation therapy.

17. The method of claim 16, wherein applying the electrically conductive layer includes affixing one or more metal plates to the second side of the insulative body.

18. The method of claim 11, further comprising covering the portion of the cable assembly that is within the set of grooves with one or more cover plates such that the portion of the cable assembly is disposed between the one or more cover plates and the second side of the base structure.

19. The method of claim 11, further comprising applying an overmold material on the first side of the base structure and the portion of the cable assembly that is within the set of grooves, wherein the overmold material conforms to a contour of the cable assembly and encases the cable assembly between the base structure and the overmold material.

20. An implantable medical device (IMD) comprising:
a pulse generator; and
a lead comprising a lead body, a cable assembly, and a shocking electrode, wherein the cable assembly electrically and mechanically connects the lead body to the shocking electrode, and the lead body extends to the pulse generator, the pulse generator configured to power the shocking electrode, via the lead body and the cable assembly, to deliver high-voltage shocks for defibrillation therapy,
wherein the shocking electrode includes a base structure that has an oblong cross-sectional shape including a first side and a second side that is opposite the first side, the base structure including a set of grooves defined along the first side, wherein a portion of the cable assembly is loaded into the grooves of the set in a side-loading direction.
